(19) United States
(12) Patent Application Publication
(10) Pub. No.: US 2006/0085059 A1
(43) Pub. Date: Apr. 20, 2006

(54) Stent having a bridge structure

(76) Inventors: Michael Ehrlinspiel, Weingarten (DE); Erik Flaxmeier, Karlsbad (DE); Alexander Lange, Karlsruhe (DE)

Correspondence Address: MCDONALD HOPKINS CO., LPA, 2100 BANK ONE CENTER, 600 SUPERIOR AVENUE, E., CLEVELAND, OH, 44114-2653, US

(21) Appl. No.: 11/081,852

(22) Filed: Mar. 16, 2005

(30) Foreign Application Priority Data
Mar. 16, 2004 (DE) 102004012837.5-43

(51) Int. Cl. A61F 2/06

(52) U.S. Cl. 623/1.15

(57) ABSTRACT

In a stent intended for implantation in a living body and having a bridge structure in which at least two bridges are coupled to one another at at least one node region on at least one of the bridges near the node region, the section modulus of the bridge varies along the length thereof, and the stresses arising at the node region upon deformation of the stent are distributed in the longitudinal direction of the bridge.

### CROSS-REFERENCE TO RELATED APPLICATIONS

This application claims priority from German Patent Application No. 10 2004 012 837.5-43 filed on Mar. 16, 2004.

### FIELD OF INVENTION

The present invention relates generally to stents intended for implantation in a living body, and in particular, to an intraluminal stent, and having a bridge structure in which at least two bridges are coupled to one another at at least one node region. The present invention also relates generally to a method for producing such a stent.

### BACKGROUND

Stents of this kind are used to protect against collapse or occlusion of channels in living bodies, for example blood vessels, esophagus, urethra or renal ducts, by expansion of their tubular bridge structure inside the channel. They also serve as carriers for medicaments in channels of the body and thus permit local therapy inside the channel.

The bridge structure of such stents is composed of a large number of bridges that are in each case connected to one another at node regions and delimit individual cells arranged alongside one another. By widening of the individual cells, the bridge structure as a whole can be expanded, and in some stents, can also be reduced in size again by making the cells smaller. The bridges thus form connection elements between the node regions that are substantially stiff and make a large contribution to the supporting action of a stent.

To ensure that the stent bears on the channel wall, it has to be able to expand radially in the channel. Additionally, in the expanded state, the stent must be able to fulfill its support function. The aim, therefore, is to design a stent optimally in terms of its deformation behavior and in terms of the resulting elongation and stress.

### SUMMARY OF THE INVENTION

An object of the invention is to make available a stent having a bridge structure that permits the greatest possible diameter ratio between the expanded state and the compressed state. In this way it should be possible to provide access into very small vessels and also ensure support of very large vessels.

According to the invention, the object is achieved with a stent in which at at least one of the bridges near the node region, the section modulus of the bridge varies along the length thereof, and the stresses arising at the node region upon deformation of the stent are distributed in the longitudinal direction of the bridge. According to the invention, the object is also achieved by a method as claimed in claim 9. Advantageous developments of a stent according to the invention and of the method are set out in the dependent claims.

In known stents, although it is likewise possible in principle to select a relatively large ratio of size between the nonexpanded state and the expanded state, the stents, however, increasingly lose their supporting force, or so-called radial force, in the expanded state. The radial force is, however, an important attribute for the use of stents.

The invention is based on the knowledge that with an increasing ratio of the size of stents, greater deformations of the bridge structure also occurs. This in turn induces greater stress in the material used. If the stresses exceed given material limits, which can generally be elongation at break and stress at break, this leads to damage of the respective element within the bridge structure of the stent. In addition, the deformed element is subject, during use, to an alternating permanent load, which, if the maximum specific to the material is exceeded, causes premature fatigue of the structural part.

To avoid this in the stent according to the invention, the induced stresses are relatively low, even at a relatively high deformation rate. Additionally, the strength of the bridge structure is comparatively great after the deformation, for example after an expansion. This bridge structure according to the invention can therefore withstand a relatively large number of alternate deformations.

According to the invention, the maximum stresses occurring are reduced by the stresses being distributed uniformly into the bridge structure of the stent. The deformation energy is thus shifted from the regions of greatest load to regions of less load.

FIG. 1 shows a section of a bridge structure 10 of a known stent 12, in which bridges 14 are provided near the node regions 16 with tapers 18 for distributing stresses within the bridge structure 10. The tapers 18 are intended to shift deformation energy from the regions of greatest load to regions where there is less load. Since the structure at the tapers takes up more deformation energy because of its reduced cross section, it relieves the inner sides of the bend points that, without tapers, are the regions where load is greatest. However, the bridge structure as a whole is weakened by the tapers 18.

The maximum stress in the stent generated by a bending moment is dependent on the section modulus of the structural part in the corresponding cross section, with a given external bending moment. The result of this is that a targeted effect on the section modulus of a stent near its node regions can influence the maximum stresses arising. The principle of the taper influences the maximum stress in the surface layer through an actual narrowing of a bridge. A narrowed bridge, however, has a disadvantageous effect when a stent is loaded, because the structure is strongly stressed by the plastic deformation in the tapered region. The deformation energy then concentrates on a relatively small material volume.

Upon alternate flexural loading of a tapered bridge on an expanded stent, caused by the usual contractions in a blood vessel, the tapered area is subjected, not only to a remaining primary stress, but also to an alternating stress. The smaller the primary stress caused by the plastic deformation, the greater the superposed alternating stress can be.

By contrast, the invention optimizes the bridge structure in terms of a reduction in induced stresses and in terms of the attainable strength after a deformation of the bridge structure. The principle according to the invention means that the stresses arising in the deformed areas are not reduced in their entirety but instead are distributed in a targeted way into other structural areas. For this purpose, the section modulus of the deformed structure is influenced in a deliberate manner.

To achieve this variation in section modulus according to the invention, the at least one bridge near the node region is designed along its length with different sizes of cross-sectional areas transverse to the longitudinal axis of the bridge. In this context, the longitudinal axis of the bridge means that axis of the bridge that extends essentially from one node region at one end of the bridge to the node region at the opposite end of the bridge. The associated cross-sectional area can also be designated as a projection of the cross section to the lever arm of the acting bending moment. According to the invention, the projection is varied in such a way that the section modulus is greater some distance away from the insides of curves of the bridge than it is in these curves themselves. In this way, deformation energy is in turn shifted from the region of greatest load to at least partially into the bridge.

Moreover, according to a preferred embodiment, the at least one bridge near the node region is designed along its length with substantially identical cross-sectional areas transverse to its main line. In this context, the main line is that (imaginary) line along which the bridge in question extends. This line is in particular curved when the bridge itself is curved or bent. Since, according to the invention, the cross-sectional area of the bridge transverses this main line, it is always comparatively large. Therefore, weakening caused by tapers, as can occur in the prior art, is substantially avoided.

Particularly, viewed in the jacket surface of the stent, the width of the at least one bridge according to the invention, at least near the node region, is substantially the same size along the length thereof. In this way, compared to a tapered bridge, the volume taken up by plastic deformation energy is greater. In this way, the primary stress remaining after the plastic deformation is smaller, since the same amount of energy is distributed across a greater volume. The alternating load that can be taken up is greater by this amount.

The stated advantages are particularly evident in a stent according to the invention in which the at least one bridge is designed near the node region, with an undulated shape along its length. With this shape, the stresses that arise are deliberately distributed into other areas of the bridge structure, without the stresses in the deformed areas being substantially reduced in their entirety.

The stent according to the present invention is provided overall with a bridge structure in which each individual bridge is designed with an undulated shape along its entire length extending between two node regions. The undulated shape of the at least one bridge can easily be produced by a punching or laser-welding process, by it being formed or cut out in the jacket surface of the stent. To ensure that the desired stress distribution in the case of loading is especially uniform, the undulated shape of the at least one bridge should moreover have alternating curves with substantially identical radii of curvature.

According to the invention, a method for producing a stent is also proposed, with the following steps: forming a bridge structure with at least two bridges and a node region arranged between them for coupling the bridges to one another, and forming at least one of the bridges near the node region in such a way that its section modulus varies along the length of the bridge, and the stresses arising at the node region upon deformation of the stent are distributed in the longitudinal direction of the bridge.

According to a preferred embodiment of the invention, in the formation step, the at least one bridge near the node region is designed along its length with different sizes of cross-sectional areas transverse to the longitudinal axis of the bridge.

Preferably, in the formation step, the at least one bridge near the node region is designed along its length with substantially identical cross-sectional areas transverse to its main line.

Also preferably, in the formation step, viewed in the jacket surface of the stent, the width of the at least one bridge, at least near the node region, is substantially the same size along its length.

Most preferably, in the formation step, the at least one bridge is designed, near the node region, with an undulated shape along its length.

More preferably, in the formation step, the at least one bridge is designed with an undulated shape along its entire length extending between two node regions.

The undulated shape of the at least one bridge is preferably formed in the jacket surface of the stent.

Preferably, the undulated shape of the at least one bridge is designed with alternating curves with in particular substantially identical radii of curvature.

### DESCRIPTION OF THE DRAWINGS

Objects and advantages together with the operation of the invention may be better understood by reference to the following detailed description taken in connection with the following illustrations, wherein:

FIG. 1 shows greatly enlarged partial views of bridge structures of known stents,

FIG. 2 shows an enlarged plan view with an enlarged detail of the bridge structure of a stent according to an embodiment of the present invention, and

FIG. 3 shows an enlarged plan view of a single bridge of the bridge structure according to FIG. 2, with simulated stress regions.

### DETAILED DESCRIPTION OF THE INVENTION

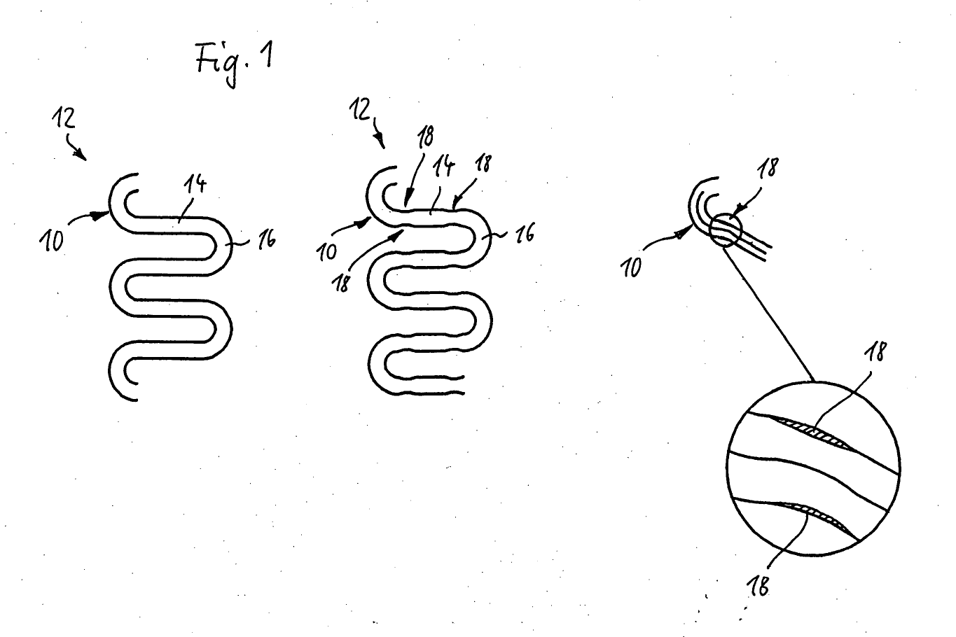

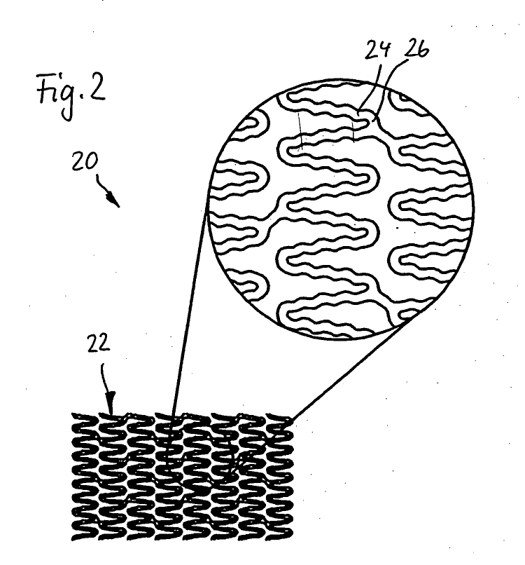

In contrast to FIG. 1 already discussed above, FIG. 2 shows a stent 20 according to an embodiment of the present invention with a bridge structure 22 in which bridges 24 are coupled to one another at node regions 26.

The bridges 24 are designed with an undulated shape along their entire length extending between two node regions 26, said undulated shape having been formed from a process such as cutting it out by a laser cutting process from a thin-walled material of a jacket surface of the stent 20. The undulated shape is designed with a number of bulges as a sequence of concave and convex arches or alternating radius elements. In the illustrative embodiment shown, such concave and convex arches are formed in succession on a single bridge 24. There can be between 3 and 12 such concave and convex arches, and in particular between 5 and 10 arches. The undulated shape of the bridges 24 can be provided in some areas of the stent 20, i.e., not all bridges 24 are provided with an undulated shape and/or the undulated shape is provided only at one area of the individual bridge 24. Thus, the bridges 24 can provide a distribution of the stresses arising during widening of the stent 20 preferably in those areas that are subjected to the greatest stress during widening of the stent 20.

Alternatively, or in addition, the stent can be produced by a punching method. The stent 20 is in this case can be produced from stainless steel or cobalt/chromium/tantalum alloys. The stent 20 is preferably widened by means of a widening device, for example a balloon catheter, in the body. Materials that can generally be used include tantalum, niobium or cobalt alloys. However, it is also conceivable to have stents made from other materials, for example polymers, self-degradable materials (e.g., lactic acid materials or derivatives), and stents made of other self-expandable materials or (preferably temperature-dependent) shape-memory materials.

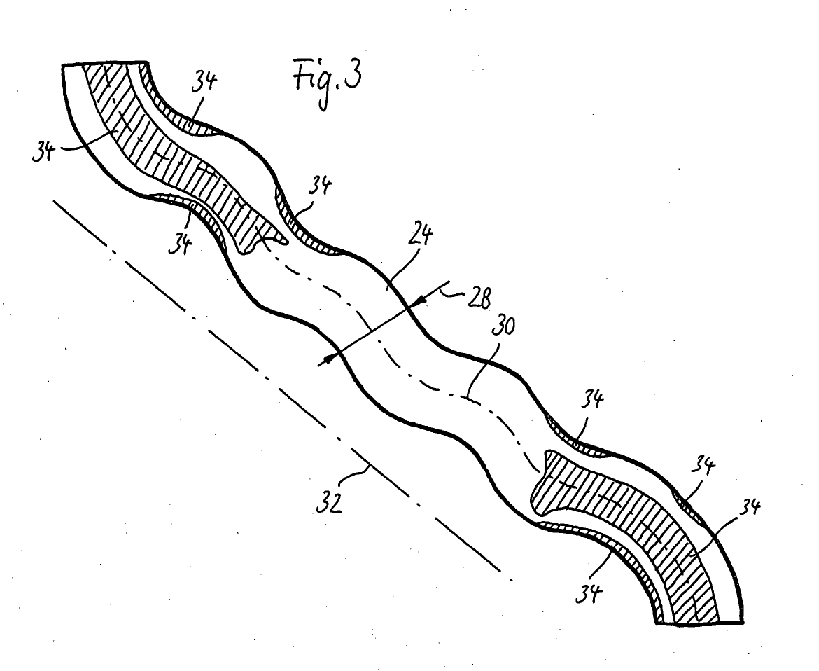

As can be seen in particular from FIG. 3, viewed in the jacket surface of the stent 20, the width 28 of each bridge 24 is substantially the same size along the length thereof. Moreover, because the thickness of the material of the jacket surface of the stent 20 is also substantially the same overall, each bridge 24 is designed along its length with substantially identical cross-sectional areas transverse to its main line 30. Viewed along the length of the bridge 24, by contrast the cross-sectional areas transverse to the longitudinal axis 32 of the bridge 24 are of different sizes.

In this way, on each bridge 24, in particular near the associated node region 26, the section moduli of the bridge 24 are varied along the length thereof, and the stresses arising at the node region 26 upon deformation of the stent 20 are distributed in the longitudinal direction of the bridge at least in some parts or some areas. Although stresses near the node region 26 are thus not reduced in their entirety, they are however distributed in a targeted manner into other structural areas of the bridge structure 22, as can be seen from the areas of increased stress 34 shown in a simplified manner in FIG. 3.

The stent 20 according to the invention or a preferred embodiment thereof can be produced both from tubular material and also from flat material. In the latter case the stent subsequently being rolled up, welded and/or finished. The stent 20 can also be produced by means of laser cutting, laser removal, photochemical etching and/or erosion. The stent 20 can also be produced with the stent structure being made in an at least partially expanded form, and the stent then being reduced in size to a compressed shape for insertion into the catheter, for example, before then being at least partially expanded again in the body.

Embodiments of the present invention have been described above and, obviously, modifications and alternations will occur to others upon a reading and understanding of this specification. In addition, the method of production described above is not limited to the order in which the steps above are recited. The claims as follows are intended to include all modifications and alterations insofar as they come within the scope of the claims or the equivalents thereof.

Having thus described the invention, we claim:

1. A stent for implantation into a living body comprising:
a first bridge having a length;
a second bridge having a length;
a node region coupling said first bridge to said second bridge;
a section modulus varying along a portion of the length of either said first bridge or said second bridge; and
wherein upon deformation of the stent, stresses arising at said node region are distributed in a longitudinal direction of said bridges in a targeted way.

2. The stent of claim 1, wherein a portion of at least one of said bridges has different sizes of cross-sectional areas transverse to the longitudinal axis of said bridge.

3. The stent of claim 1, wherein a portion of the at least one bridge has substantially identical cross-sectional areas transverse to its main line.

4. The stent of claim 1, wherein the at least one bridge includes a width and wherein the width of the at least one bridge is substantially of equal size along a portion of the length thereof.

5. The stent of claim 1, wherein the at least one bridge has an undulated shape along a portion of its length.

6. The stent of claim 5, wherein the at least one bridge has an undulated shape along its entire length.

7. The stent of claim 6, wherein the undulated shape of the at least one bridge is formed in a jacket surface of the stent.

8. The stent of claim 7, wherein the undulated shape of the at least one bridge has alternating curves with substantially identical radii of curvature.

9. A method for producing a stent comprising:
forming a first bridge having a length;
forming a second bridge having a length;
coupling said first bridge to said second bridge at a node region;
forming a section modulus varying along a portion of the length of either said first bridge or said second bridge, wherein upon deformation of the stent, stresses arising at said node region are distributed in a longitudinal direction of said bridges in a targeted way.

10. The method of claim 9, wherein at least one of said bridges includes a longitudinal axis and wherein a portion of the at least one bridge has different sizes of cross-sectional areas transverse to the longitudinal axis of said bridge.

11. The method of claim 10, wherein a portion of the at least one bridge has substantially identical cross-sectional areas transverse to its main line.

12. The method of claim 11, wherein the at least one bridge includes a width and wherein the width is substantially of equal size along a portion of the length of the at least one bridge.

13. The method of claim 12, wherein the at least one bridge has an undulated shape along a portion of its length.

14. The method of claim 13, wherein the at least one bridge has an undulated shape along its entire length.

15. The method of claim 14, wherein the undulated shape of the at least one bridge is formed in a jacket surface of the stent.

16. The method of claims 15, wherein the undulated shape of the at least one bridge has alternating curves with substantially identical radii of curvature.

17. A stent comprising:
a first bridge having a length and a main line;
a second bridge having a length and a main line;
a node region coupling said first bridge to said second bridge;
a section modulus varying along a portion of the length of either said first bridge or second bridge;
wherein either of said first bridge or said second bridge has a substantially identical cross-sectional area transverse to the main line thereof; and
wherein upon deformation of the stent, stresses arising at said node region are distributed in a longitudinal direction of said bridges in a targeted way.

18. The stent of claim 17, wherein at least one of said bridges includes a width and wherein the width of the at least one bridge is substantially of equal size along a portion of the length thereof.

19. The stent of claim 17, wherein either the first bridge or the second bridge has an undulated shape along its entire length.

20. The stent of claim 19, wherein the undulated shape has alternating curves with substantially identical radii or curvature.
